(19) United States
(12) Patent Application Publication
(10) Pub. No.: US 2025/0201362 A1
(43) Pub. Date: Jun. 19, 2025

(54) NAVIGATING HISTORICAL HEALTHCARE INFORMATION

(71) Applicant: Express Scripts Strategic Development, Inc., St. Louis, MO (US)

(72) Inventor: Peter Lewis, Fairfax City, VA (US)

(21) Appl. No.: 18/986,394

(22) Filed: Dec. 18, 2024

Related U.S. Application Data
(60) Provisional application No. 63/611,735, filed on Dec. 18, 2023.

(51) Int. Cl. G16H 10/60; G06Q 50/00

(52) U.S. Cl. CPC G16H 10/60; G06Q 50/01

(57) ABSTRACT

An example computer system includes memory hardware configured to store historical patient information, and processor hardware configured to execute instructions including accessing historical patient information associated with a patient, identifying a plurality of events in the historical patient information that satisfy one or more criteria, processing, by a first large language model (LLM), the plurality of events that satisfy the one or more criteria, to generate a historical health output associated with the patient, generating, for display in a graphical user interface, one or more interactive medical tiles associated with the plurality of events that satisfy the one or more criteria, according to the historical health output associated with the patient, receiving a selection of one of the interactive medical tiles, and presenting, in the graphical user interface, the plurality of events according to an organization criterion in response to receiving the selection of one of the interactive medical tiles.

### CROSS-REFERENCE TO RELATED APPLICATION

This application claims the benefit and priority of U.S. Provisional Application No. 63/611,735, filed on Dec. 18, 2023. The entire disclosure of the above application is incorporated herein by reference.

### FIELD

The present disclosure relates systems and methods for navigating historical healthcare information.

### BACKGROUND

Patient medical records are managed in a variety of ways. Certain medical records include prescriptions for medicinal drugs (also referred to as drug prescriptions) written or provided by various healthcare professionals. Pharmacists usually process these prescriptions to dispense the corresponding medications to a patient.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

An example computer system includes memory hardware configured to store historical patient information and computer-executable instructions, and processor hardware configured to execute the computer-executable instructions. The computer-executable instructions include accessing historical patient information associated with a patient, identifying a plurality of events in the historical patient information that satisfy one or more criteria, processing, by a first large language model (LLM), the plurality of events that satisfy the one or more criteria, to generate a historical health output associated with the patient, generating, for display in a graphical user interface, one or more interactive medical tiles associated with the plurality of events that satisfy the one or more criteria, according to the historical health output associated with the patient, receiving a selection of one of the interactive medical tiles, and presenting, in the graphical user interface, the plurality of events according to an organization criterion in response to receiving the selection of one of the interactive medical tiles.

In some examples, the one or more interactive medical tiles are displayed in a health summary format on the graphical user interface, and the health summary format is specified according to the historical health output from the LLM.

In some examples, the health summary format includes at least one of a chatbot conversation textual output format, a health journey timeline output format, a periodic update textual output forma, and a social media update output format.

In some examples, the health summary format includes the chatbot conversation textual output format, and the chatbot conversation textual output format includes at least one health summary comment displayed in response to a user input prompt.

In some examples, the health summary format includes the health journey timeline output format, and the health journey timeline output format includes multiple health events displayed in consecutive timeline by event date, next to an electronic health record of the patient corresponding to the health event.

In some examples, the health summary format includes the periodic update textual output format, and the periodic update textual output format includes a summary one or more health events for the patient occurring within a last day, a last week and a last month.

In some examples, accessing historical patient information associated with the patient includes accessing multiple electronic health records of the patient from an electronic health record database.

In some examples, accessing historical patient information associated with the patient includes accessing at least one of community and support data associated with the patient, data acquired from one or more wearable devices of the patient, environmental data corresponding to an environment of the patient, or demographic data of the patient.

In some examples, processing, by the first large language model (LLM), the plurality of events that satisfy the one or more criteria, includes assigning a higher weight to the multiple electronic health records of the patient compared to data obtained from other sources.

In some examples, processing, by the first large language model (LLM), the plurality of events that satisfy the one or more criteria, includes processing the plurality of events to determine at least one of a healthcare milestone achieved by the patient, a healthcare setback experienced by the patient, healthcare goal progress achieved by the patient, an ally support event associated with the patient, or a healthcare decision made by the patient.

In some examples, processing, by the first large language model (LLM), the plurality of events that satisfy the one or more criteria, includes processing the plurality of events to determine at least one of a healthcare need defined by the patient, a healthcare goal defined by the patient, a best practice related to healthcare for the patient, a provider guidance item for the patient, or a treatment plan for a health condition of the patient.

In some examples, the computer-executable instructions include receiving input from the patient that includes one or more keywords related to an intent associated with one or more of the plurality of events, processing, by the first large language model (LLM), the input from the patient to generate a prompt for a second LLM, and processing the prompt by the second LLM together with the historical patient information to generate a response to the input.

In some examples, executable instructions further include processing the historical patient information to predict a set of inquiries associated with the plurality of events, and receiving, as part of the input, a selection of an individual inquiry of the set of inquiries.

In some examples, the first LLM comprises an artificial neural network, and wherein the second LLM comprises an artificial neural network. In some examples, the input comprises a document inquiry, and the computer-executable instructions further include receiving a medical document as part of the input, and processing the medical document by the second LLM to predict a set of intents associated with the medical document.

In some examples, the computer-executable instructions further include generating a query based on content of the medical document and an individual intent of the set of intents, obtaining information corresponding to the query, and presenting the information in the graphical user interface.

In some examples, the computer-executable instructions further include processing the historical patient information by an artificial neural network to select the plurality of events, the artificial neural network being trained using training data to identify events that satisfy the one or more criteria.

In some examples, the computer-executable instructions further include accessing training data comprising training patient information and corresponding ground truth collections of events in the training patient information that satisfy the one or more criteria, processing, by the artificial neural network, the training patient information to estimate a plurality of events, computing a deviation between the plurality of events and the corresponding ground truth collections of events, and updating one or more parameters of the artificial neural network based on the computed deviation.

An example method includes accessing historical patient information associated with a patient, identifying a plurality of events in the historical patient information that satisfy one or more criteria, processing, by a first large language model (LLM), the plurality of events that satisfy the one or more criteria, to generate a historical health output associated with the patient, generating, for display in a graphical user interface, one or more interactive medical tiles associated with the plurality of events that satisfy the one or more criteria, according to the historical health output associated with the patient, receiving a selection of one of the interactive medical tiles, and presenting, in the graphical user interface, the plurality of events according to an organization criterion in response to receiving the selection of one of the interactive medical tiles.

An example non-transitory computer-readable medium comprising non-transitory computer-readable instructions for performing operations comprising accessing historical patient information associated with a patient, identifying a plurality of events in the historical patient information that satisfy one or more criteria, processing, by a first large language model (LLM), the plurality of events that satisfy the one or more criteria, to generate a historical health output associated with the patient, generating, for display in a graphical user interface, one or more interactive medical tiles associated with the plurality of events that satisfy the one or more criteria, according to the historical health output associated with the patient, receiving a selection of one of the interactive medical tiles, and presenting, in the graphical user interface, the plurality of events according to an organization criterion in response to receiving the selection of one of the interactive medical tiles.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a block diagram of an example patient management platform, according to some examples.

FIG. 2 is an example database that may be deployed within the system of FIG. 1, according to some examples.

FIG. 3 is a block diagram of an example patient management platform that may be deployed within the system of FIG. 1, according to some examples.

FIGS. 4 and 5 are block diagrams of example user interfaces of the patient management platform, according to some examples.

FIG. 6 is a flowchart illustrating example operations of the patient management platform, according to some examples.

FIG. 7 is a block diagram illustrating an example software architecture that may be used in conjunction with various hardware architectures described herein.

FIG. 8 is a block diagram illustrating components of a machine, according to some examples.

FIG. 9 is a functional block diagram of an example neural network that can be used for the inference engine or other functions (e.g., engines) as described herein to produce a predictive model.

FIG. 10 is a functional block diagram of an example system for navigating historical healthcare information.

In the drawings, reference numbers may be reused to identify similar and/or identical elements.

### DETAILED DESCRIPTION

Example methods and systems for a patient management platform are provided. Specifically, the methods and systems provide large language model (LLM)-driven automated healthcare. The methods and systems access historical patient information associated with a patient. The methods and systems identify a plurality of events in the historical patient information that satisfies one or more criteria. The methods and systems generate, for display in a graphical user interface, an interactive medical tile associated with the plurality of events that satisfies the one or more criteria. The methods and systems receive input that selects the interactive medical tile. The methods and systems present, in the graphical user interface, the plurality of events according to an organization criterion in response to receiving the input that selects the interactive medical tile. The present systems and methods can dynamically, predictively, and autonomously perform tasks for a patient via inputs received from the client devices. The present systems and methods can relate data about a user, e.g., a patient, in unique ways and provide the data to a user at a time when it is needed by the user for a specific use case, e.g., everyday life, medical provider visit, intervention, medical procedure, prescription renewal, prescription refill, medication recommendation, surgery, post operative care, prescribed lifestyle changes, and the like.

In various examples, the LLMs can be trained on information about healthcare rules, healthcare systems, and benefits systems, which can include policies, accounts (HSAs), etc. and patient information such as the plan in which they are enrolled, patient health records, user (e.g., patient, provider, or regulatory) activity interacting with the health products and services, interactions with health and benefits providers, financial info, etc. This wholistic data can enable the LLM to predict important tasks, user intent/needs, and suggest responses or tasks to complete (by the user through their device, automatically on their behalf or by the provider through their device, automatically on their behalf). The examples provided herein describe embodiments to present and implement this computer implemented experience.

In the following description, for purposes of explanation, numerous specific details are set forth in order to provide a thorough understanding of the examples. It will be evident, however, to one of ordinary skill in the art that examples of the disclosure may be practiced without these specific details.

Patients spend a considerable amount of time seeking medical information. For example, patients spend a great deal of time researching information about new drugs they are prescribed, verifying medical benefits, paying for healthcare, booking medical appointments, and so forth. Sometimes, the information gathered by the patients is wholly inaccurate or not updated, which can have disastrous consequences for patients relying on such information. As a result, patients are burdened with having to navigate multiple pages of information to look up the appropriate answers to the questions they seek and, even then, may still need to get to a final resolution. This wastes a great deal of time and resources that can be devoted to other tasks.

The disclosed techniques provide systems and methods to automate and assist patients with gathering medical information and performing medical-related tasks. The disclosed techniques leverage LLMs to automate such tasks. The disclosed techniques access historical patient information associated with a patient. The disclosed techniques identify a plurality of events in the historical patient information that satisfies one or more criteria. The disclosed techniques generate, for display in a graphical user interface, an interactive medical tile associated with the plurality of events that satisfies the one or more criteria. The disclosed techniques receive input that selects the interactive medical tile. The disclosed techniques present, in the graphical user interface, the plurality of events according to an organization criterion in response to receiving the input that selects the interactive medical tile.

In some cases, the disclosed techniques process patient information to predict a set of inquiries related to a patient and receive input from the patient that selects an individual inquiry from the set of inquiries, and that includes one or more keywords related to an intent associated with the selected individual inquiry. Examples of the disclosed techniques process, by a first LLM, the input from the patient to generate a prompt for a second LLM and process the prompt by the second LLM together with the patient information to generate a response to the individual inquiry.

As a result, a great deal of time and resources are saved, and the user need not have to navigate through many pages of information to generate responses to answers patients seek. This saves time and reduces the amount of resources needed to accomplish a task.

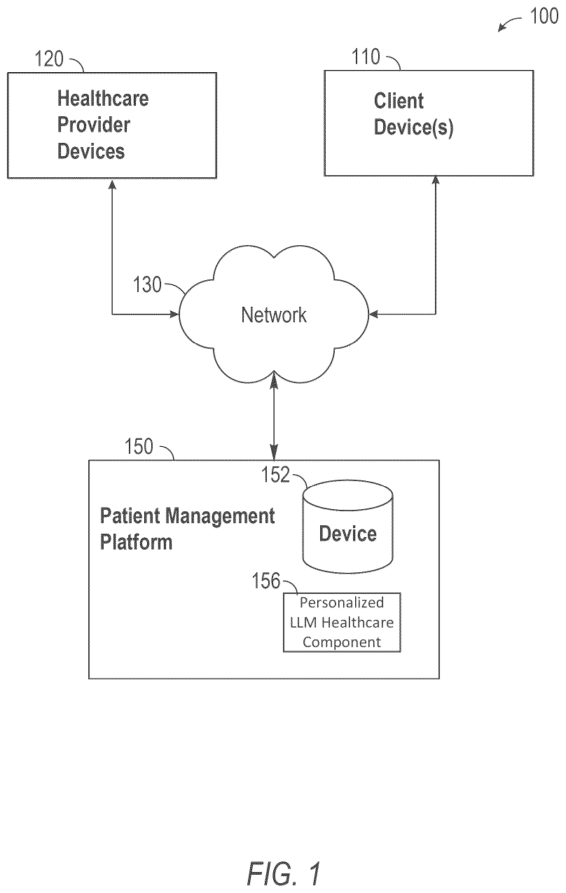

FIG. 1 is a block diagram showing an example of patient management system 100, according to some examples. The patient management system 100 includes one or more client devices 110, one or more healthcare provider devices 120, and a patient management platform 150 that are communicatively coupled over a network 130 (e.g., Internet, telephony network).

The term “client device” may refer to any machine that interfaces to a communications network (such as network 130) to access the patient management platform 150. The client device 110 may be but is not limited to, a mobile phone, desktop computer, laptop, portable digital assistant (PDA), smartphone, wearable device (e.g., a smartwatch), tablets, ultrabooks, netbooks, laptops, multi-processor systems, microprocessor-based or programmable consumer electronics, game consoles, set-top boxes, or any other communication device that a user may use to access a network or the patient management platform 150. These devices are dedicated machines to performing tasks or instructions described herein to executed the functions and methodologies described herein.

In some cases, the patient management platform 150 is accessible over a global communication system, e.g., the Internet or the World Wide Web. In such instances, the patient management platform 150 hosts a website accessible to the client devices 110. Upon accessing the website, the client devices 110 provide secure login credentials, which are used to access a profile associated with the login credentials and one or more patient profiles or patient information. As used herein, patient information includes any medical information associated with a patient, including one or more medicinal drug prescriptions, prior medical insurance claims that were approved or denied, one or more electronic health records or medical health records, patient health information, patient demographic information, prior bloodwork results, prior results of non-bloodwork tests, medical history, medical provider notes in the electronic health record, intake forms completed by the patient, patient in-network insurance coverage, patient out-of-network insurance coverage, patient location, and/or one or more treatment preferences. One or more user interfaces associated with the patient management platform 150 are provided over the Internet via the website to the client devices 110. The user interfaces may include set locations or fixed locations where they displays the patient data.

Healthcare provider devices 120 can include the same or similar functionality as client devices 110 for accessing the patient management platform 150. In some cases, the healthcare provider devices 120 are used by “internal” users. Internal users are medical professionals, such as medical personnel, physicians, healthcare professionals, clinicians, healthcare providers, health-related coaches, pharmacy benefit manager (PBM) operators, pharmacists, specialty pharmacy operators or pharmacists, or the like that are associated with, certified by, or employed by one or more organizations that provide the patient management platform 150. In some cases, the healthcare provider devices 120 are used by “external” users. Moreover, data that can be input into the large language models to produce an output on the user device can be sourced from the provider devices.

The healthcare provider devices 120 are used to access the patient management platform 150 and view many records associated with many different patients (or users associated with client devices 110) and their respective patient information. Different authorization levels can be associated with other users to control which records the users have access to. In some instances, only records associated with those patients to which a given user is referred are made accessible and available to the given user device. Sometimes, a first user can refer a patient or records associated with the patient to a second user. In such circumstances, the second user becomes automatically authorized to access and view the patient's records that the first user referred. The user interfaces on the healthcare provider devices 120 may include set locations or fixed locations where it displays the patient data.

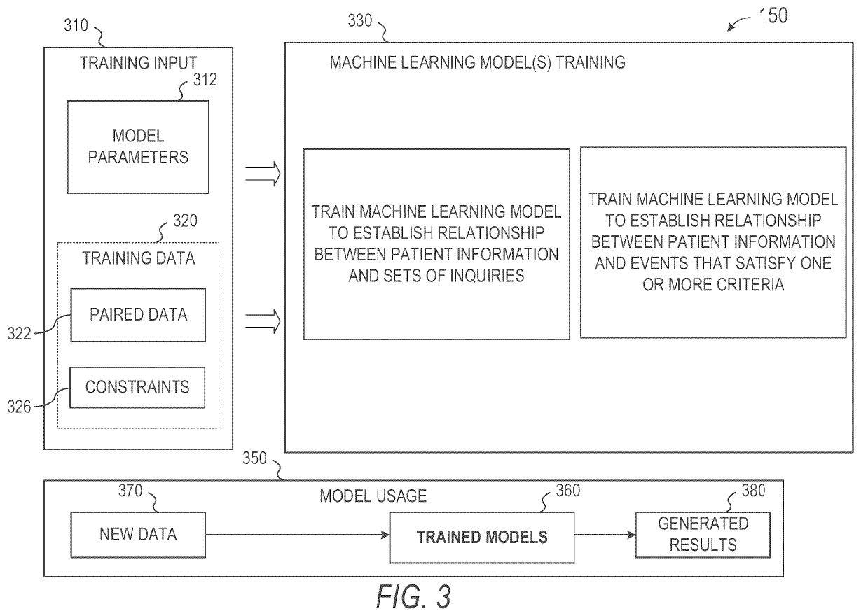

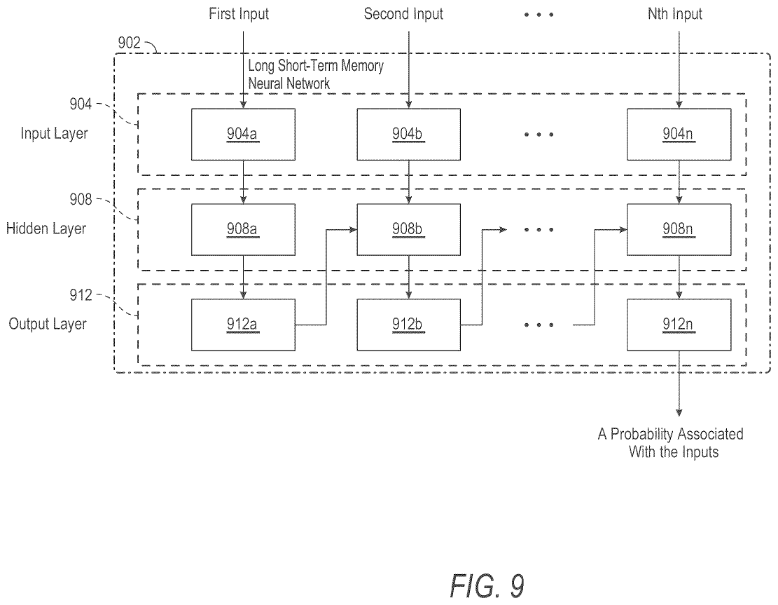

In some examples, the patient management platform 150 (specifically the personalized LLM healthcare component 156) can implement one or more machine learning models, such as one or more neural networks (discussed below in connection with FIGS. 3 and 9). The patient management platform 150 can use machine learning models to simplify and expedite the management of patient information and particularly to generate or predict responses associated with prior authorizations for prescriptions, or other healthcare related issues. The present systems and methods can provide predicted interactions related to inquiries regarding claims, medical care, payment, and the like. Particularly, the patient management platform 150 can be accessed by the healthcare provider devices 120. The patient management platform 150 can use machine learning models to simplify and expedite the management of patient information and particularly to generate

[symbol]

In some examples, the patient management platform 150 accesses historical patient information associated with a patient. The patient management platform 150 identifies a plurality of events in the historical patient information that satisfies one or more criteria. The patient management platform 150 generates, for display in a graphical user interface, an interactive medical tile associated with the plurality of events that satisfies the one or more criteria. The patient management platform 150 receives input that selects the interactive medical tile. The patient management platform 150 presents, in the graphical user interface, the plurality of events according to an organization criterion in response to receiving the input that selects the interactive medical tile.

In some cases, the patient management platform 150 can dynamically, predictively, and autonomously perform tasks for a patient via inputs received from the client devices 110. The patient management platform 150 can leverage one or more LLMs and other generative AIs to process patient information for a patient and provide personalized and predicted inquiries the patient may be interested in selecting. This can be performed when an event and/or interactive medical tile is selected by input received from the client device 110 of the patient. Once an individual inquiry is selected, input from the client devices 110 can be received that provides one or more keywords (e.g., in pictorial, image, text, and/or voice). A first LLM is applied to the selected inquiry, and one or more keywords are used to generate a prompt for a second LLM. The second LLM can perform one or more tasks based on the prompt to provide results or responses for presentation to the patient on the client device 110. A respective machine learning model, such as a generative AI machine learning model, can implement each LLM.

Generative artificial intelligence (AI) is a term that may refer to any artificial intelligence that can create new content from training data. For example, generative AI can produce text, images, video, audio, code, or synthetic data that are similar to the original data but not identical. In some cases, generative AI can include or implement large language models (LLMs). The generative AI and/or LLMs receive a prompt (including instructions) and a set of data to process based on the prompt. The generative AI and/or LLMs process the data in accordance with the instructions of the prompt and generate an output that includes modifications of the set of data based on prior knowledge of the generative AI and/or LLMs.

Some of the techniques that may be used in generative AI are:

Convolutional Neural Networks (CNNs): CNNs are commonly used for image recognition and computer vision tasks. They are designed to extract features from images using filters or kernels that scan the input image and highlight important patterns. CNNs may be used in object detection, facial recognition, and autonomous driving applications.

Recurrent Neural Networks (RNNs): RNNs are designed for processing sequential data, such as speech, text, and time series data. They have feedback loops that allow them to capture temporal dependencies and remember past inputs. RNNs may be used in speech recognition, machine translation, and sentiment analysis applications.

Generative adversarial networks (GANs): These models consist of two neural networks: a generator and a discriminator. The generator tries to create realistic content that can fool the discriminator, while the discriminator tries to distinguish between real and fake content. The two networks compete with each other and improve over time. GANs may be used in image synthesis, video prediction, and style transfer applications.

Variational autoencoders (VAEs): These models encode input data into a latent space (a compressed representation) and then decode it back into output data. The latent space can be manipulated to generate new variations of the output data. They may use self-attention mechanisms to process input data, allowing them to handle long text sequences and capture complex dependencies.

Transformer models: These models use attention mechanisms to learn the relationships between different parts of input data (such as words or pixels) and generate output data based on these relationships. Transformer models can handle sequential data, such as text or speech, and non-sequential data, such as images or code.

In generative AI examples, the prediction/inference data that is output includes trend assessment and predictions, translations, summaries, image or video recognition and categorization, natural language processing, face recognition, user sentiment assessments, advertisement targeting and optimization, voice recognition, or media content generation, recommendation, and personalization.

The machine learning model (or generative AI) can have access to a wide variety of patient information that is stored in the database 152. The machine learning model can process a wide variety of patient information and extract a portion of the patient information based on a set of prompts. After collecting the portion of the patient information, the patient management platform 150 can communicate with one or more entities (e.g., other LLMs or human medical professionals).

In some examples, the patient management platform 150 trains the personalized LLM healthcare component 156 by performing training operations, including obtaining a batch of training data that includes a first collection of medical information associated with a first set of ground truth inquiries. The personalized LLM healthcare component 156 processes the first collection of medical information by the machine learning model to generate an estimated set of inquiries and computes a loss based on a deviation between the estimated set of inquiries and the first set of ground truth inquiries. The personalized LLM healthcare component 156 updates one or more parameters of the personalized LLM healthcare component 156 based on the computed loss. The patient management platform 150 repeats these training operations for multiple batches of the training data and completes training of the personalized LLM healthcare component 156 when a stopping condition/criterion is reached.

In some examples, the patient management platform 150 trains the personalized LLM healthcare component 156 by performing training operations, including obtaining a batch of training data that includes training patient information and corresponding ground truth collections of events in the training patient information that satisfy the one or more criteria. The personalized LLM healthcare component 156 processes the training patient information by the machine learning model to generate an estimated set of events and computes a loss based on a deviation between the estimated set of events that satisfy the one or more criteria and the ground truth collections of events. The personalized LLM healthcare component 156 updates one or more parameters of the personalized LLM healthcare component 156 based on the computed loss. The patient management platform 150 repeats these training operations for multiple batches of the training data and completes training of the personalized LLM healthcare component 156 when a stopping condition/criterion is reached.

The network 130 may include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network. The coupling may be a Code Division Multiple Access (CDMA) connection, a Global System for Mobile Communications (GSM) connection, or other cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth-generation wireless (5G) networks, Universal Mobile Telecommunications System (UMTS), High-Speed Packet Access (HSPA), Worldwide Interoperability for Microwave Access (WiMAX), Long Term Evolution (LTE) standard, others defined by various standard setting organizations, other long-range protocols, or other data transfer technology.

The healthcare provider devices 120 can access pharmacy claims, medical data (e.g., medical information 230 stored in database 152), laboratory data, and the like for one or more patients that the healthcare provider devices 120 are authorized to view. This patient information 210 can be maintained in a database 152 by the patient management platform 150 or in a third-party database accessible to the patient management platform 150 and/or the healthcare provider devices 120.

In some examples, the client devices 110 and the patient management platform 150 can be communicatively coupled via an audio call (e.g., VOIP, Public Switched Telephone Network, cellular communication network, etc.) or via electronic messages (e.g., online chat, instant messaging, text messaging, email, and the like). While FIG. 1 illustrates a single client device 110 and a single healthcare provider device 120, it is understood that a plurality of such devices can be included in the system 100 in other embodiments.

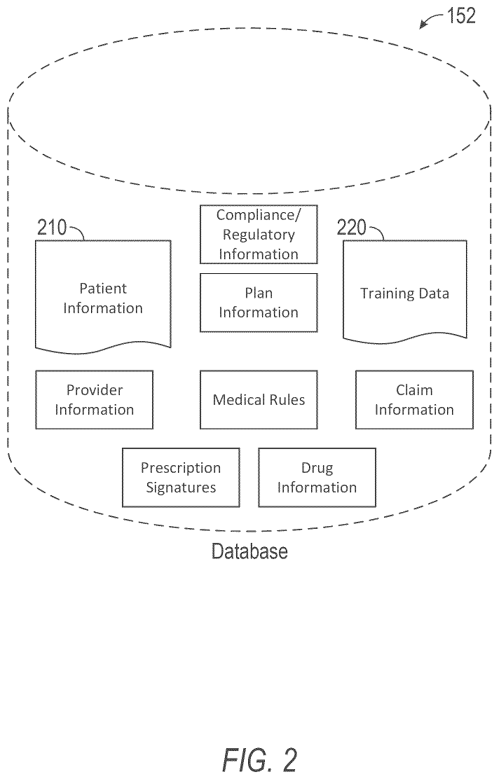

FIG. 2 is an example database 152 that may be deployed within the system of FIG. 1, according to some examples. The database 152 includes patient information 210 and training data 220. The patient information 210 can be generated or accessed by the patient management platform 150. For example, the patient management platform 150 can access one or more patient records from one or more sources, including pharmacy claims, benefits information, prescribing physician information, dispensing information (e.g., where and how the patient obtains their current medications), medicinal drug prescriptions, prescription signatures, demographic data, prescription information including dose quantity and interval, and input from a patient received via a user interface presented on the client device 110 and so forth. The patient management platform 150 can collect this information from the patient records and generate a patient features vector that includes this information.

The training data 220 is used to train the personalized LLM healthcare component 156 implemented by patient management platform 150 to generate inquiries and responses automatically and/or to identify a plurality of events in historical patient information that satisfies one or more criteria.

In some examples, the personalized LLM healthcare component 156 implements an AI bot architecture in which multiple AI bots and/or LLMs operate together and are orchestrated to perform medical tasks for a patient.

In some examples, the personalized LLM healthcare component 156 can access historical patient information associated with a patient. The personalized LLM healthcare component 156 can process the historical patient information to identify different groups, collections, or subsets of medical events that satisfy one or more criteria. For example, a first collection of medical events can be determined to satisfy a first criterion and a second collection of medical events can be determined to satisfy a second criterion that is different from the first criterion. The personalized LLM healthcare component 156 can associate each collection of medical events with a respective interactive medical tile in a graphical user interface.

In some examples, the one or more criteria include events related to a same medical condition associated with the patient. Specifically, the personalized LLM healthcare component 156 can process the historical patient information to identify a set of events that relate to the same medical condition. For example, the personalized LLM healthcare component 156 can determine that the patient has been diagnosed with melanoma during a doctor visit that is included among the set of events. The personalized LLM healthcare component 156 can also identify an ultrasound, x-ray, MRI, or CT scan performed on the patient in association with the melanoma diagnosis. The personalized LLM healthcare component 156 can identify one or more prescriptions in the historical patient information that relate to the treatment of melanoma. The personalized LLM healthcare component 156 can determine that each of these events is associated with the same diagnosis of melanoma. In such cases, the personalized LLM healthcare component 156 groups all of the events in association with the same medical condition and can generate a first interactive medical tile that represents this group of medical events.

In some examples, the one or more criteria include events associated with a medical event that is on a list of significant medical events. The list of significant medical events comprises changes in activity levels of the patient, completion of a physical medical examination, and hospital stay. For example, the personalized LLM healthcare component 156 can process the historical patient information to identify a specific event (e.g., a medical claim) that was received from an emergency room of a hospital. The personalized LLM healthcare component 156 can search the list of significant medical events and determine that emergency room visits are included among the list of significant medical events. The personalized LLM healthcare component 156 can also identify one or more prescriptions in the historical patient information that were generated by an entity associated with the emergency room visit or hospital. The personalized LLM healthcare component 156 can identify a medical diagnosis associated with the emergency room visit and search the patient information for other events associated with the medical diagnosis. The personalized LLM healthcare component 156 groups all of the events in association with the medical event that is determined to be significant and can generate a second interactive medical tile that represents this group of medical events.

In some examples, the one or more criteria include medical records associated with a current location of a user device of the patient. For example, the personalized LLM healthcare component 156 can access location information from client device 110 of the patient. The personalized LLM healthcare component 156 can search for medical facilities that are within a threshold distance of the location of the client device 110 of the patient. In response to identifying a medical facility that is within the threshold distance, the personalized LLM healthcare component 156 obtains events stored in the patient information that correspond to the medical facility. The personalized LLM healthcare component 156 groups all of the events in association with the medical facility and can generate a third interactive medical tile that represents this group of medical events.

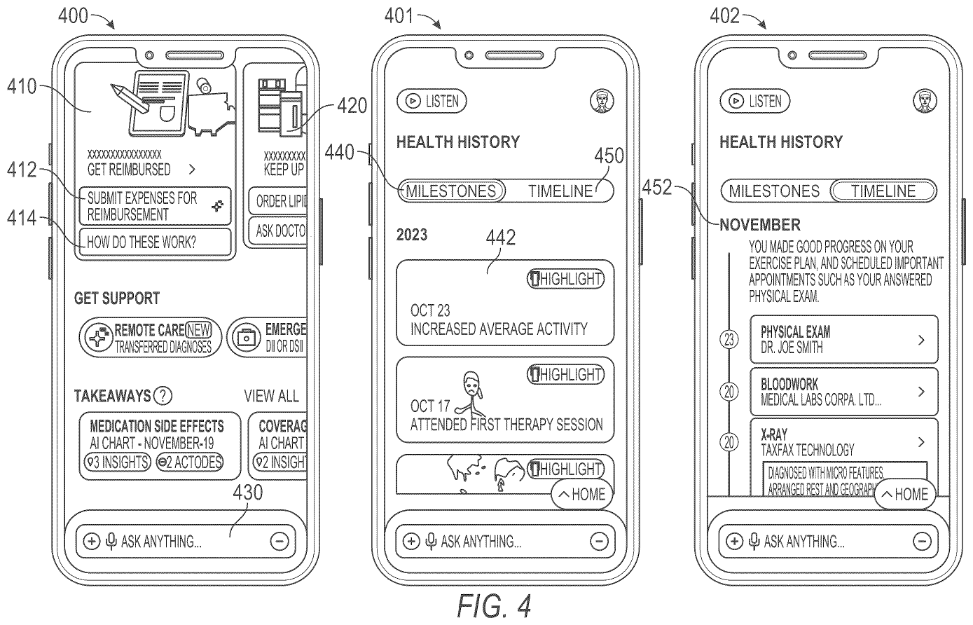

For example, as shown in FIG. 4, a graphical user interface 400 can be presented to a patient on the client device 110. The graphical user interface 400 can include a plurality of interactive medical tiles 410 and 420. The interactive medical tile 410 can represent a first collection of events of the patient information that correspond to a first set of criteria. The interactive medical tile 420 can represent a second collection of events of the patient information that correspond to a second set of criteria. The personalized LLM healthcare component 156 can process patient information to generate a predictive set of inquiries for each of the first collection of events and the second collection of events. These predictive sets of inquiries can be used in conjunction with one or more user-supplied keywords to generate a prompt for one or more LLMs to perform a specific operation. The graphical user interface 400 can sometimes present the predictive set of inquiries 412 and 414 inside of the corresponding interactive medical tile. For example, the interactive medical tile 410 can include a first set of inquiries 412 and 414 that were generated by the LLM. The interactive medical tile 420 can include a second set of inquiries that were generated by the LLM. The list of inquiries can be populated based on a machine learning model prediction of what the user is most likely interested in inquiring about. In some cases, a predicted selection of one of the components can be highlighted for the patient.

Once an individual inquiry is selected based on input from the client device 110 of the patient, the personalized LLM healthcare component 156 can automatically generate a response. In some cases, the personalized LLM healthcare component 156 receives one or more keywords that are input in the keyword entry region 430. The keyword entry region 430 can be adjacent to a region of the graphical user interface 400 in which the interactive medical tile 410 is presented. The keyword entry region 432 can receive input textually, verbally, and/or via one or more images/videos. For example, input can be received that selects a microphone option. In response, verbal input is collected and transcribed into one or more keywords. The personalized LLM healthcare component 156 can then generate a prompt, such as using a first LLM, based on the selected inquiry and the one or more keywords provided in the keyword entry region 430. Specifically, once the component is selected, the client devices 110 receives input from the patient that includes one or more keywords (in various forms) that are processed by the personalized LLM healthcare component 156 to generate a response in an automated, efficient, and fast manner.

In some examples, the keyword entry region 430 can receive input from the patient that includes a certain document or set of documents (e.g., a receipt, medical bill, explanation of benefits, and so forth). In response to receiving the certain document or set of documents, the personalized LLM healthcare component 156 processes the certain document or set of documents to predict a set of inquiries the patient may be seeking answers to in relation to the certain document or set of documents and/or based on the events associated with a selected interactive medical tile 410. The personalized LLM healthcare component 156 can access patient information for the patient to predict one or more of the set of inquiries. The personalized LLM healthcare component 156 can present the set of inquiries on a graphical user interface to allow the patient to quickly and easily select an inquiry to which a response is being sought. The personalized LLM healthcare component 156 can then communicate with one or more other systems or LLM to obtain the response. Namely, the personalized LLM healthcare component 156 can process the document or set of documents to retrieve information that corresponds to the selected inquiry and to identify which one or more entities (e.g., LLMs or medical professionals) need to be contacted to obtain a response. For example, if the user selects an amount on a bill, the personalized LLM healthcare component 156 can contact an insurance provider of the patient and provide an identifier of the bill, which has been extracted from the documents as well as the amount specified on the bill. The personalized LLM healthcare component 156 can receive or generate an explanation associated with the amount or contact a medical provider to dispute the amount automatically.

For example, the graphical user interface 400 can include a document upload region. The client device 110 of the patient can be used to submit or upload one or more documents in the document upload region. In response to receiving the documents, the personalized LLM healthcare component 156 processes the documents using one or more LLMs to identify what types of documents have been uploaded. The one or more LLMs can also use patient information to predict what inquiries the patient may have about the uploaded documents. Then, the graphical user interface 400 can be presented to the user with the predicted inquiries and can allow the user to supply one or more keywords to specify an intent associated with the documents, such as via the keyword entry region 430. In some cases, the interactive medical tile 410 and the interactive medical tile 420 can be presented according to an organization criterion, such as based on importance or significance of the events associated with each tile and/or chronologically.

The graphical user interface 400 can receive input that selects the interactive medical tile 410. In response, the graphical user interface 400 navigates the user to a graphical user interface 401. In the graphical user interface 401, different subset of the medical events associated with the interactive medical tile 410 can be presented according to different types of organization criteria. For example, a first subset of medical events can be presented according to a first organization criterion, such as milestones. A second subset of medical events can be presented according to a second organization criterion, such as a timeline.

In response to receiving input that selects a first option 440 corresponding to the first organization criterion, the first subset of medical events 442 can be presented. The first subset of medical events 442 can correspond to events that are labeled as milestones or that include attributes that correspond to milestones.

In response to receiving input that selects a second option 450 corresponding to the second organization criterion, the second subset of medical events 452 can be presented in a graphical user interface 402. The second subset of medical events 452 can present a chronological timeline of the subset of medical events associated with the interactive medical tile 410. For example, a first medical event (e.g., doctor visit) can be identified and determined to correspond to a first timepoint and a second medical event (prescription fulfillment) can be identified and determined to correspond to a first timepoint. As such, the personalized LLM healthcare component 156 can present the first medical event chronologically higher in a list of events than the second medical event. Any event listed in among the first subset of medical events 442 and/or the second subset of medical events 452 can be selected by input from the client device 110 to obtain further information. In some cases, the personalized LLM healthcare component 156 can receive one or more keywords from the patient while the first subset of medical events 442 and/or the second subset of medical events 452 are presented. The personalized LLM healthcare component 156 can generate a prompt that identifies the medical events being listed and the one or more keywords that were received and provides that prompt to another LLM for generating a response to a query associated with the one or more keywords.

In some cases, the personalized LLM healthcare component 156 can receive input that selects an individual event listed among the first subset of medical events 442 and/or the second subset of medical events 452. In response, the personalized LLM healthcare component 156 can predict a set of inquiries related to the event that is selected. For example, the personalized LLM healthcare component 156 can process the selected event by a machine learning model. The machine learning model can be trained to predict a set of inquiries for the selected event and output the predicted set of inquiries. The personalized LLM healthcare component 156 can receive the predicted set of inquiries and present the set of inquiries in a graphical user interface along with information for the selected event.

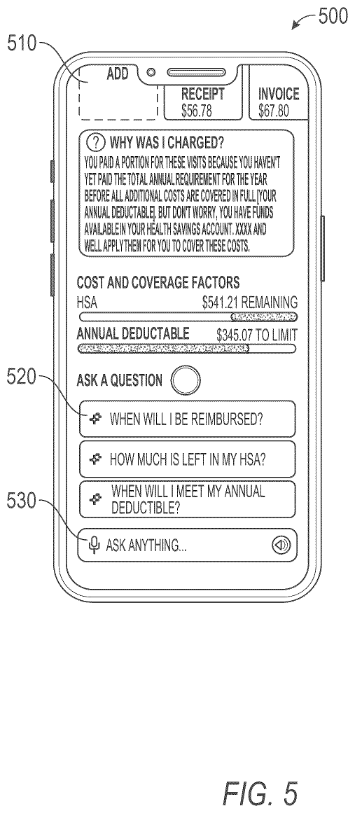

For example, as shown in the graphical user interface 500 of FIG. 5, input may have been received that selects a particular medical event from the graphical user interface 401 or graphical user interface 402. The graphical user interface 500 can present a set of information 510 associated with the particular medical event, such as information retrieved from the patient information corresponding to the event. The graphical user interface 500 can receive the predicted set of inquiries from the personalized LLM healthcare component 156 and present the set of inquiries in a region 520. The set of inquiries can be ranked based on importance. Input can be received from the patient that selects an individual inquiry from the region 520. Additionally, one or more keywords can be received in the region 530. The individual inquiry along with the one or more keywords can be processed by an LLM to generate a prompt. The prompt can be processed by another machine learning model and/or LLM to generate a response for presentation to the patient on the client device 110.

In some example embodiments described herein, one or more large language models (LLMs) may be used to access one or more sources of data associated with a patient, such as an electronic health record (EHR), and generate a historical summary output based on processing the data with the LLM(s). For example, one or more LLM models may access community and support data associated with a patient, wearables data, environmental data, patient data, demographic data, EHRs of the patient, etc., and process the data related to life events and context (e.g., milestones, setbacks, goal progress, allies, decision, etc.), user definitions (e.g., needs, goals, etc.), and next steps (e.g., best practices, provider guidance, treatment plans, etc.), to generate a historical summary output. The historical summary output may have any suitable specified output, including a “story” format that is engaging for a user, such as a chatbot conversation, a health journey timeline, a periodic update, a social media update, etc.

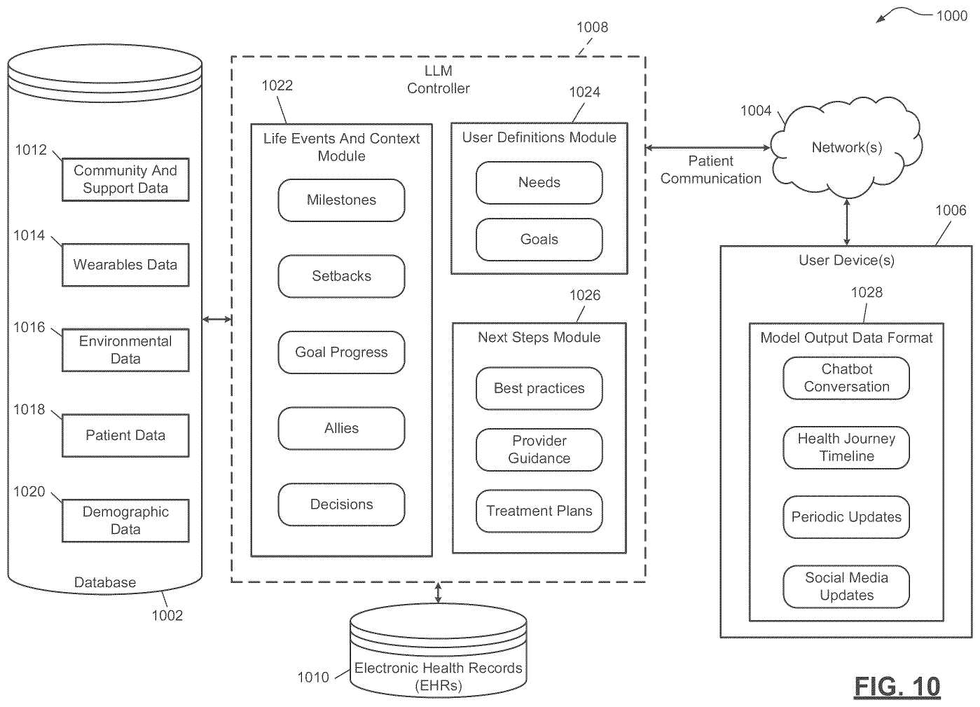

FIG. 10 is a functional block diagram of an example system 1000 for navigating historical healthcare information, including a database 1002 and an electronic health record (EHR) database 1010 (which may be part of the same database or different databases). While the system 1000 is generally described as being deployed in a computer network system, the database 1002, the EHR database 1010 and/or components of the system 1000 may otherwise be deployed (for example, as a standalone computer setup or in the patient management platform 150). The system 1000 may include a desktop computer, a laptop computer, a tablet, a smartphone, a server, cloud data storage and processing, etc.

As shown in FIG. 10, the database 1002 stores community and support data 1012, wearables data 1014, environmental data 1016, patient data 1018, and demographic data 1020. In various implementations, the database 1002 may store other types of data as well. The community and support data 1012, wearables data 1014, environmental data 1016, patient data 1018, and demographic data 1020 may be located in different physical memories within the database 1002, such as different random access memory (RAM), read-only memory (ROM), a non-volatile hard disk or flash memory, etc. For example, some data may be stored on servers of a third party vendor.

In some implementations, the community and support data 1012, wearables data 1014, environmental data 1016, patient data 1018, and demographic data 1020 may be located in the same memory (such as in different address ranges of the same memory). In various implementations, the community and support data 1012, wearables data 1014, environmental data 1016, patient data 1018, and demographic data 1020 may each be stored as structured data in any suitable type of data store.

The community and support data 1012 may include any suitable data regarding input from caregivers of a patient, family or friends of a patient, etc. For example, the community and support data 1012 may include feedback about a health status of the patient from friends or family, observations from a caregiver about a condition of the patient, etc.

The wearables data 1014 may include any suitable data obtained from tracking devices worn by the patient, such as smartwatches, cellular phones, heart rate monitors, step counters, sleep monitors, etc. The wearables data 1014 may provide information about step counts of the patient, meals eaten by the patient, sleep of the patient, activity of the patient, etc.

The environmental data 1016 may include any suitable data indicating current or past environmental conditions, such as weather, news, traffic, etc. The patient data 1018 may include any suitable data associated with activity of the patient, such as audio or written journal entries, interactions with a medical information application, etc.

The demographic data 1020 may include any suitable data indicative of demographics of the patient, such as patient background information, age, gender, phase of life, marital status, family status, relational status, etc. Other example embodiments may include other types of data and data sources. An example of relational status is described in U.S. Pat. No. 11,039,014, which hereby incorporated by reference. The relational status may be determined using assistance from an LLM. The demographic data may also be accessed via public databases, patients logging in or approving access to private database sources, etc.

As mentioned above, the EHR database 1010 may be a part of the database 1002, or a separate database. The EHR database 1010 may include any suitable electronic health records of the patient, such as patient appointments (e.g., diagnoses from a historical appointment, treatment plans, provider notes, referrals, etc.). The EHR record data may include test results for the patient, prescriptions, treatments, etc.

The LLM controller 1008 may include one or more modules for using LLMs to process data from the database 1002 and the EHR database 1010 data, and generate health summary outputs for a patient. For example, a health summary output from an LLM may be communicated to a patient according to patient communication needs, preferences, styles, etc. (such as an interactive phone application, an email, a text message, an automated notification or a phone call), and displayed on a user device 1006 associated with the patient. The user device 1006 may include any suitable user device for displaying text and receiving input from a user, including a desktop computer, a laptop computer, a tablet, a smartphone, etc. In various implementations, the user device 1006 may access the database 1002 or the LLM controller 1008 directly, or may access the database 1002 or the LLM controller 1008 through one or more networks 1004. Example networks may include a wireless network, a local area network (LAN), the Internet, a cellular network, etc.

FIG. 10 illustrates the LLM controller as including examples of a life events and context module 1022, a user definitions module 1024, and a next steps module 1026. Other example embodiments may include more or less modules.

The life events and context module 1022 may use one or more LLMs to process data from the database 1002 and the EHR database 1010 to generate outputs associated with life events of the patient. For example, an LLM may process data associated with the patient to identify milestones accomplished by the patient, such as a first therapy session, attending a specified streak of physical checkup appointments, completing a treatment, achieving a new weight goal, etc.

The LLM may process data associated with the patient to identify setbacks and lessons learned, such as overcoming an injury, identifying times where a patient has higher or lower energy for accomplishing tasks, etc. The LLM may process data to identify progress toward goals, such as reducing weight towards a specified goal, competing a number of treatments in a sequence treatment plan, etc. This may include insights and benefits gained by the patient as the progress towards the goal.

The LLM may process data to identify allies of the patient, such as new caregivers, friends, family, etc., which have connected with the patient online, are monitoring or celebrating health success or progress of the patient, etc. The LLM may process data to identify health decisions made by the patient, such as selecting a care plan for a specified health condition, selecting a health care provider, etc.

The user definitions module 1024 may include suitable needs, goals, etc., for the patient. These needs and goals may be defined specifically by the patient, or suggested based on LLM processing of data associated with the patient. For example, needs may include conditions set by the patient, or health conditions that need to be treated for the patient, patient preferences (such as communication preferences, health treatment preferences, appointment or scheduling preferences, etc.). The goals may include patient progress in treatments outlines, fitness goals of the patient, lifestyle goals, relationship goals, etc.

The next steps module 1026 may include any suitable data about next health care steps for the patient, based on LLM processing of data associated with the patient. For example, the next seps may be based on best practices corresponding to treating health conditions or preventative healthcare for the patient, provider guidance related to the patient, specified treatment plans for a patient, etc.

As mentioned above, the output of the LLM(s) may include historical health summaries that are provided to the user in an engaging story format. For example, output of the LLM(s) may be displayed on a user device 1006, such as a health navigation application for a cellular phone or computer. The model output data format 1028 may include any suitable format for displaying an engaging story of health care information to the patient, such as a chatbot conversation.

The model output data format 1028 may include a health journey timeline, which displays milestones achieved by the patient over time on their health journey. In some examples, each milestone may be displayed as an individual medical tile on a user interface of the user device 1006 that the user may interact with, or may be grouped with other milestones in a single medical tile.

In some examples, health information may be displayed along side data from the EHR of the patient, such as a congratulations on attending a first therapy session, or completing a series of treatments, next to an EHR record and date of the therapy session or the last completed treatment appointment.

The model output format 1028 may include periodic updates, such as a summary of health updates for the patient for the day, within a last week, within a last month, etc. The output may be in the form of an audio update, a textual update, etc. Other example formats could include an update suitable for posting on social media, any other suitable story or narrative formats that engage the user while providing helpful information about the patient's health history, etc.

In some examples, different types of data may be weighted differently for processing by the LLMs. For example, EHR records from the EHR database may be assigned a higher weight than other data types from the database 1002, where the EHR records provide the most direct support of the patient's health history. When available, wearables data 1014 may be weighted higher when processing data by LLMs for milestones or goal progress of the patient, and community and support data 1012 may be weighted higher when processing data related to allies of the patient.

FIG. 3 is a block diagram of an example service of patient management platform 150 that may be deployed within the system of FIG. 1, according to some examples. Training input 310 includes model parameters 312 and training data 320 (e.g., training data 220 (FIG. 2)) which may include paired training data sets 322 (e.g., input-output training pairs) and constraints 326. Model parameters 312 stores or provides the parameters or coefficients of corresponding ones of machine learning models. During training, these parameters 312 are adapted based on the input-output training pairs of the training data sets 322. After the parameters 312 are adapted (after training), the parameters are used by trained models 360 to implement the trained machine learning models on a new set of data 370.

Training data 320 includes constraints 326, which may define the constraints of a given patient information feature. The paired training data sets 322 may include sets of input-output pairs, such as pairs of a plurality of patient information features and features of inquiries associated with the patient information. Some components of training input 310 may be stored separately at a different off-site facility or facility than other components. The paired training data sets 322 may include sets of input-output pairs, such as pairs of a plurality of medical events features and features of events that satisfy one or more criteria.

Machine learning model(s) training 330 trains one or more machine learning techniques based on the sets of input-output pairs of paired training data sets 322. For example, model training 330 may train the machine learning (ML) model parameters 312 by minimizing a loss function based on one or more ground-truth data.

The ML models can include any one or combination of classifiers, LLMs, or neural networks, such as an artificial neural network, a convolutional neural network, an adversarial network, a generative adversarial network, a deep feed-forward network, a radial basis network, a recurrent neural network, a long/short term memory network, a gated recurrent unit, an autoencoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, and the like.

Particularly, a first ML model of the ML models can be applied to a training batch of patient information features to estimate or generate a prediction of inquiries associated with the patient information. In some implementations, a derivative of a loss function is computed based on a comparison of the estimated prediction of the inquiries and the ground truth inquiries, and parameters of the first ML model are updated based on the computed derivative of the loss function. The result of minimizing the loss function for multiple sets of training data trains adapts, or optimizes the model parameters 312 of the corresponding first ML model. In this way, the first ML model is trained to establish a relationship between a plurality of training patient information and ground-truth inquiries.

A second ML model of the ML models can be applied to a training batch of medical event features to estimate or generate a prediction of medical events that satisfy one or more criteria. In some implementations, a derivative of a loss function is computed based on a comparison of the estimated prediction of the events and the ground truth events that satisfy the one or more criteria, and parameters of the second ML model are updated based on the computed derivative of the loss function. The result of minimizing the loss function for multiple sets of training data trains adapts, or optimizes the model parameters 312 of the corresponding second ML model. In this way, the second ML model is trained to establish a relationship between a plurality of medical events and ground-truth events that satisfy one or more criteria.

After the machine learning models are trained, new data 370, including historical patient information features, are received and/or derived by the patient management platform 150. The first/second trained machine learning model may be applied to the new data 370 to generate results 380, including a prediction of inquiries and/or events that satisfy one or more criteria.

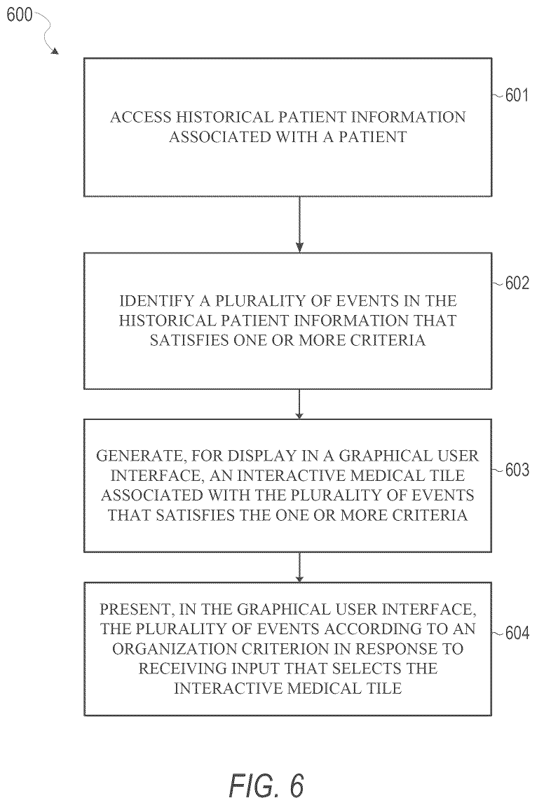

FIG. 6 is a flowchart illustrating example operations and methods of the patient management platform 150 in performing a method or process 600, according to some examples. The process 600 may be embodied in computer-readable instructions for execution by one or more processors such that the operations of the process 600 may be performed in part or in whole by the functional components of the system 100 (e.g., personalized LLM healthcare component 156); accordingly, the process 600 is described below by way of example with reference thereto. However, in other examples, at least some of the operations of the process 600 may be deployed on various other hardware configurations. Some or all of the operations of process 600 can be in parallel, out of order, or entirely omitted.

At operation 601, the system 100 accesses historical patient information associated with a patient, as discussed above.

At operation 602, the system 100 identifies a plurality of events in the historical patient information that satisfies one or more criteria, as discussed above.

At operation 603, the system 100 generates, for display in a graphical user interface, an interactive medical tile associated with the plurality of events that satisfies the one or more criteria, as discussed above.

At operation 604, the system 100 presents, in the graphical user interface, the plurality of events according to an organization criterion in response to receiving input that selects the interactive medical tile, as discussed above.

### EXAMPLES

Example 1. A method comprising: accessing historical patient information associated with a patient; identifying a plurality of events in the historical patient information that satisfies one or more criteria; generating, for display in a graphical user interface, an interactive medical tile associated with the plurality of events that satisfies the one or more criteria; receiving input that selects the interactive medical tile; and presenting, in the graphical user interface, the plurality of events according to an organization criterion in response to receiving the input that selects the interactive medical tile.

Example 2. The method of Example 1, wherein the one or more criteria comprise events related to a same medical condition associated with the patient.

Example 3. The method of any one of Examples 1-2, wherein the one or more criteria comprise events associated with a medical event that is on a list of significant medical events.

Example 4. The method of Example 3, wherein the list of significant medical events comprises changes in activity levels of the patient, completion of a physical medical examination, and hospital stay.

Example 5. The method of any one of Examples 1-4, further comprising presenting a plurality of interactive medical tiles in the graphical user interface comprising the interactive medical tile, each of the plurality of medical tiles corresponding to a different collection of events that satisfies the one or more criteria.

Example 6. The method of any one of Examples 1-5, further comprising: processing the historical patient information by an artificial neural network to select the plurality of events, the artificial neural network being trained using training data to identify events that satisfy the one or more criteria.

Example 7. The method of Example 6, further comprising: accessing training data comprising training patient information and corresponding ground truth collections of events in the training patient information that satisfy the one or more criteria; processing, by the artificial neural network, the training patient information to estimate a plurality of events; computing a deviation between the plurality of events and the ground truth collections of events; and updating one or more parameters of the artificial neural network based on the computed deviation.

Example 8. The method of any one of Examples 1-7, further comprising: grouping a first subset of the plurality of events in a first organization criterion; and grouping a second subset of the plurality of events in a second organization criterion.

Example 9. The method of Example 8, wherein the first organization criterion comprises milestones, and wherein the second organization criterion comprises a timeline.

Example 10. The method of Example 9, further comprising: presenting the second subset of the plurality of events in chronological order.

Example 11. The method of any one of Examples 8-10, further comprising: receiving first input that selects a first option corresponding to the first organization criterion; and presenting the first subset of the plurality of events in response to receiving the first input.

Example 12. The method of Example 11, further comprising: receiving second input that selects a second option corresponding to the second organization criterion; and presenting the second subset of the plurality of events in response to receiving the second input.

Example 13. The method of any one of Examples 1-12, wherein the one or more criteria comprise medical records associated with a current location of a user device of the patient.

Example 14. The method of any one of Examples 1-13, further comprising: receiving input from the patient that includes one or more keywords related to an intent associated with one or more of the plurality of events; processing, by a first large language model (LLM), the input from the patient to generate a prompt for a second LLM; and processing the prompt by the second LLM together with the patient information to generate a response to the input.

Example 15. The method of Example 14, further comprising: processing the patient information to predict a set of inquiries associated with the plurality of events; and receiving, as part of the input, a selection of an individual inquiry of the set of inquiries.

Example 16. The method of any one of Examples 14-15, wherein the first LLM comprises an artificial neural network, and wherein the second LLM comprises an artificial neural network.

Example 17. The method of any one of Examples 14-16, wherein the input comprises a document inquiry, further comprising: receiving a medical document as part of the input; and processing the medical document by the second LLM to predict a set of intents associated with the medical document.

Example 18. The method of Example 17, further comprising: generating a query based on content of the medical document and an individual intent of the set of intents; obtaining information corresponding to the query; and presenting the information in the graphical user interface.

Example 19. A system comprising: one or more processors coupled to a memory comprising non-transitory computer instructions that when executed by the one or more processors perform operations comprising: accessing historical patient information associated with a patient; identifying a plurality of events in the historical patient information that satisfies one or more criteria; generating, for display in a graphical user interface, an interactive medical tile associated with the plurality of events that satisfies the one or more criteria; receiving input that selects the interactive medical tile; and presenting, in the graphical user interface, the plurality of events according to an organization criterion in response to receiving the input that selects the interactive medical tile.

Example 20. A non-transitory computer-readable medium comprising non-transitory computer-readable instructions for performing operations comprising: accessing historical patient information associated with a patient; identifying a plurality of events in the historical patient information that satisfies one or more criteria; generating, for display in a graphical user interface, an interactive medical tile associated with the plurality of events that satisfies the one or more criteria; receiving input that selects the interactive medical tile; and presenting, in the graphical user interface, the plurality of events according to an organization criterion in response to receiving the input that selects the interactive medical tile.

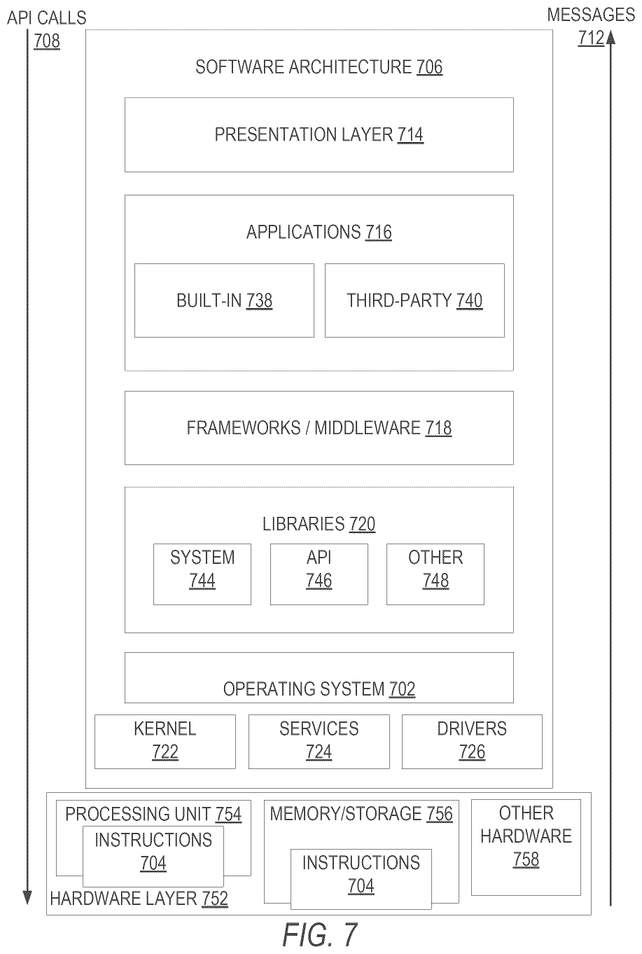

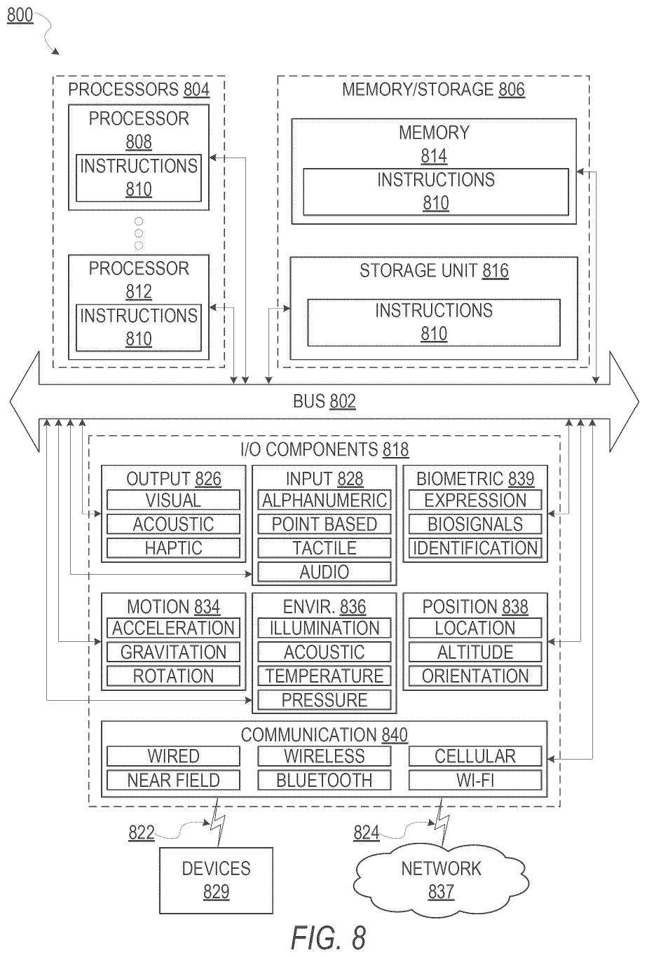

FIG. 7 is a block diagram illustrating an example software architecture 706, which may be used in conjunction with various hardware architectures herein described. FIG. 7 is a non-limiting example of a software architecture and it will be appreciated that many other architectures may be implemented to facilitate the functionality described herein. The software architecture 706 may execute on hardware such as machine 800 of FIG. 8 that includes, among other things, processors 804, memory 814, and input/output (I/O) components 818. A representative hardware layer 752 is illustrated and can represent, for example, the machine 800 of FIG. 8. The representative hardware layer 752 includes a processing unit 754 having associated executable instructions 704. Executable instructions 704 represent the executable instructions of the software architecture 706, including implementation of the methods, components, and so forth described herein. The hardware layer 752 also includes memory and/or storage devices memory/storage 756, which also have executable instructions 704. The hardware layer 752 may also comprise other hardware 758. The software architecture 706 may be deployed in any one or more of the components shown in FIG. 1. The software architecture 706 can be utilized to leverage one or more LLMs to accomplish a task associated with an inquiry presented to a patient.

In the example architecture of FIG. 7, the software architecture 706 may be conceptualized as a stack of layers where each layer provides particular functionality. For example, the software architecture 706 may include layers such as an operating system 702, libraries 720, frameworks/middleware 718, applications 716, and a presentation layer 714. Operationally, the applications 716 and/or other components within the layers may invoke API calls 708 through the software stack and receive messages 712 in response to the API calls 708. The layers illustrated are representative in nature and not all software architectures have all layers. For example, some mobile or special purpose operating systems may not provide a frameworks/middleware 718, while others may provide such a layer. Other software architectures may include additional or different layers.

The operating system 702 may manage hardware resources and provide common services. The operating system 702 may include, for example, a kernel 722, services 724, and drivers 726. The kernel 722 may act as an abstraction layer between the hardware and the other software layers. For example, the kernel 722 may be responsible for memory management, processor management (e.g., scheduling), component management, networking, security settings, and so on. The services 724 may provide other common services for the other software layers. The drivers 726 are responsible for controlling or interfacing with the underlying hardware. For instance, the drivers 726 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 720 provide a common infrastructure that is used by the applications 716 and/or other components and/or layers. The libraries 720 provide functionality that allows other software components to perform tasks in an easier fashion than to interface directly with the underlying operating system 702 functionality (e.g., kernel 722, services 724 and/or drivers 726). The libraries 720 may include system libraries 744 (e.g., C standard library) that may provide functions such as memory allocation functions, string manipulation functions, mathematical functions, and the like. In addition, the libraries 720 may include API libraries 746 such as media libraries (e.g., libraries to support presentation and manipulation of various media format such as MPREG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 720 may also include a wide variety of other libraries 748 to provide many other APIs to the applications 716 and other software components/devices.

The frameworks/middleware 718 (also sometimes referred to as middleware) provide a higher-level common infrastructure that may be used by the applications 716 and/or other software components/devices. For example, the frameworks/middleware 718 may provide various graphic user interface functions, high-level resource management, high-level location services, and so forth. The frameworks/middleware 718 may provide a broad spectrum of other APIs that may be utilized by the applications 716 and/or other software components/devices, some of which may be specific to a particular operating system 702 or platform.

The applications 716 include built-in applications 738 and/or third-party applications 740. Examples of representative built-in applications 738 may include, but are not limited to, a contacts application, a browser application, a book reader application, a location application, a media application, a messaging application, and/or a game application. Third-party applications 740 may include an application developed using the ANDROID™ or IOS™ software development kit (SDK) by an entity other than the vendor of the particular platform, and may be mobile software running on a mobile operating system such as IOS™, ANDROID™ WINDOWS® Phone, or other mobile operating systems. The third-party applications 740 may invoke the API calls 708 provided by the mobile operating system (such as operating system 702) to facilitate functionality described herein.

The applications 716 may use built-in operating system functions (e.g., kernel 722, services 724, and/or drivers 726), libraries 720, and frameworks/middleware 718 to create UIs to interact with users of the system. Alternatively, or additionally, in some systems, interactions with a user may occur through a presentation layer, such as presentation layer 714. In these systems, the application/component “logic” can be separated from the aspects of the application/component that interact with a user.

FIG. 8 is a block diagram illustrating components of a machine 800, according to some examples, able to read instructions from a machine-readable medium (e.g., a machine-readable storage medium) and perform any one or more of the methodologies discussed herein. Specifically, FIG. 8 shows a diagrammatic representation of the machine 800 in the example form of a computer system, within which instructions 810 (e.g., software, a program, an application, an applet, an app, or other executable code) for causing the machine 800 to perform any one or more of the methodologies discussed herein may be executed. For example, the instructions 810 may be executed by the system 100 to process a medicinal drug prescription document or communication by the patient management platform 150 with trained machine learning models to leverage one or more LLMs to accomplish a task associated with an inquiry presented to a patient.

As such, the instructions 810 may be used to implement devices or components described herein. The instructions 810 transform the general, non-programmed machine 800 into a particular machine 800 programmed to carry out the described and illustrated functions in the manner described. In alternative examples, the machine 800 operates as a standalone device or may be coupled (e.g., networked) to other machines. In a networked deployment, the machine 800 may operate in the capacity of a server machine or a client machine in a server-client network environment, or as a peer machine in a peer-to-peer (or distributed) network environment. The machine 800 may comprise, but not be limited to a server computer, a client computer, a personal computer (PC), a tablet computer, a laptop computer, a netbook, a STB, a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 810, sequentially or otherwise, that specify actions to be taken by machine 800. Further, while only a single machine 800 is illustrated, the term “machine” shall also be taken to include a collection of machines that individually or jointly execute the instructions 810 to perform any one or more of the methodologies discussed herein.

The machine 800 may include processors 804, memory/storage 806, and I/O components 818, which may be configured to communicate with each other such as via a bus 802. In an example embodiment, the processors 804 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 808 and a processor 812 that may execute the instructions 810. The term “processor” is intended to include multi-core processors 804 that may comprise two or more independent processors (sometimes referred to as “cores”) that may execute instructions contemporaneously. Although FIG. 8 shows multiple processors 804, the machine 800 may include a single processor with a single core, a single processor with multiple cores (e.g., a multi-core processor), multiple processors with a single core, multiple processors with multiple cores, or any combination thereof.

The memory/storage 806 may include a memory 814, such as a main memory, or other memory storage, database 152, and a storage unit 816, both accessible to the processors 804 such as via the bus 802. The storage unit 816 and memory 814 store the instructions 810 embodying any one or more of the methodologies or functions described herein. The instructions 810 may also reside, completely or partially, within the memory 814, within the storage unit 816, within at least one of the processors 804 (e.g., within the processor's cache memory), or any suitable combination thereof, during execution thereof by the machine 800. Accordingly, the memory 814, the storage unit 816, and the memory of processors 804 are examples of machine-readable media.

The I/O components 818 may include a wide variety of components to receive input, provide output, produce output, transmit information, exchange information, capture measurements, and so on. The specific I/O components 818 that are included in a particular machine 800 will depend on the type of machine. For example, portable machines such as mobile phones will likely include a touch input device or other such input mechanisms, while a headless server machine will likely not include such a touch input device. It will be appreciated that the I/O components 818 may include many other components that are not shown in FIG. 8. The I/O components 818 are grouped according to functionality merely for simplifying the following discussion and the grouping is in no way limiting. In various example embodiments, the I/O components 818 may include output components 826 and input components 828. The output components 826 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 828 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further examples, the I/O components 818 may include biometric components 839, motion components 834, environmental components 836, or position components 838 among a wide array of other components. For example, the biometric components 839 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 834 may include acceleration sensor components (e.g., accelerometer), gravitation sensor components, rotation sensor components (e.g., gyroscope), and so forth. The environmental components 836 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 838 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 818 may include communication components 840 operable to couple the machine 800 to a network 837 or devices 829 via coupling 824 and coupling 822, respectively. For example, the communication components 840 may include a network interface component or other suitable device to interface with the network 837. In further examples, communication components 840 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 829 may be another machine or any of a wide variety of peripheral devices (e.g., a peripheral device coupled via a USB).

Moreover, the communication components 840 may detect identifiers or include components operable to detect identifiers. For example, the communication components 840 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 840, such as location via Internet Protocol (IP) geo-location, location via Wi-Fi® signal triangulation, location via detecting an NFC beacon signal that may indicate a particular location, and so forth.

FIG. 9 is a functional block diagram of an example neural network 902 that can be used for the inference engine or other functions (e.g., engines) as described herein to produce a predictive model. The predictive model can identify or generate inquiries associated with patient information. In an example, the neural network 902 can be a LSTM neural network. In an example, the neural network 902 can be a recurrent neural network (RNN). The example neural network 902 may be used to implement the machine learning as described herein, and various implementations may use other types of machine learning networks. The neural network 902 includes an input layer 904, a hidden layer 908, and an output layer 912. The input layer 904 includes inputs 904a, 904b . . . 904n. The hidden layer 908 includes neurons 908a, 908b . . . 908n. The output layer 912 includes outputs 912a, 912b . . . 912n.

Each neuron of the hidden layer 908 receives an input from the input layer 904 and outputs a value to the corresponding output in the output layer 912. For example, the neuron 908a receives an input from the input 904a and outputs a value to the output 912a. Each neuron, other than the neuron 908a, also receives an output of a previous neuron as an input. For example, the neuron 908b receives inputs from the input 904b and the output 912a. In this way the output of each neuron is fed forward to the next neuron in the hidden layer 908. The last output 912n in the output layer 912 outputs a probability associated with the inputs 904a-904n. Although the input layer 904, the hidden layer 908, and the output layer 912 are depicted as each including three elements, each layer may contain any number of elements. Neurons can include one or more adjustable parameters, weights, rules, criteria, or the like.

In various implementations, each layer of the neural network 902 must include the same number of elements as each of the other layers of the neural network 902. For example, training features (e.g., collection of patient information associated with a first set of ground truth inquiries and/or collections of events and corresponding ground truth events that match one or more criteria) may be processed to create the inputs 904a-904n.

The neural network 902 may implement a first model to produce a set of inquiries. More specifically, the inputs 904a-904n can include fields of the patient information as data features (binary, vectors, factors or the like) stored in the storage device 110. The features of the patient information can be provided to neurons 908a-908n for analysis and connections between the known facts. The neurons 908a-908n, upon finding connections, provides the potential connections as outputs to the output layer 912, which determines a set of inquiries associated with the patient information.

The neural network 902 can perform any of the above calculations. The output of the neural network 902 can be used to control an LLM to retrieve the appropriate set of medical information. In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 904a is connected to each of neurons 908a, 908b . . . 908n.

Embodiments as described herein can be used with the system described in U.S. Patent No. 12, 148,014 ('014), issued 19 Nov. 2024, which is hereby incorporated by reference. For example, the present LLM for providing personalized, individual outputs 156 can reside in the digital health developer in the '014 patent. The user output 1028 from the LLM can be fed into user device, to provider devices, a pharmacy benefit manager system, a pharmacy or the like. The user output 1028 can be used, at least in part, to develop a care plan.

### Glossary

“CARRIER SIGNAL” in this context refers to any intangible medium that is capable of storing, encoding, or carrying transitory or non-transitory instructions for execution by the machine, and includes digital or analog communications signals or other intangible medium to facilitate communication of such instructions. Instructions may be transmitted or received over the network using a transitory or non-transitory transmission medium via a network interface device and using any one of a number of well-known transfer protocols. The instructions can carry a selected model that automatically copy text from a first interface and automatically identifies a target location in a second interface at which the copied data from the first interface is suggested to be copied.

“CLIENT DEVICE” in this context refers to any machine that interfaces to a communications network to obtain resources from one or more server systems or other client devices. A client device may be, but is not limited to, a mobile phone, desktop computer, laptop, PDA, smart phone, tablet, ultra-book, netbook, laptop, multi-processor system, microprocessor-based or programmable consumer electronics, game console, set-top box, or any other communication device that a user may use to access a network. In an example embodiment, the client device is capable of having two or more display that can have two or more interfaces, from which the system selects information to copy and a target location to insert the copied information on two different interfaces. In an example embodiment, the first interface is different than the second interface. The first interface can be produced a different program than the second interface. The first interface can be operating on a different database than the second interface.

“COMMUNICATIONS NETWORK” in this context refers to one or more portions of a network that may be an ad hoc network, an intranet, an extranet, a virtual private network (VPN), a LAN, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device, or other tangible media able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions (e.g., code) for execution by a machine, such that the instructions, when executed by one or more processors of the machine, cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes transient signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an ASIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine. A processor may, for example, be a CPU, a RISC processor, a CISC processor, a GPU, a DSP, an ASIC, a RFIC, or any combination thereof. A processor may further be a multi-core processor having two or more independent processors (sometimes referred to as “cores”) that may execute instructions contemporaneously.

Changes and modifications may be made to the disclosed techniques without departing from the scope of the present disclosure. These and other changes or modifications are intended to be included within the scope of the present disclosure, as expressed in the following claims.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

### Conclusion

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set. The term “non-empty set” may be used to indicate exclusion of the empty set. The term “subset” does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A computer system comprising:
memory hardware configured to store historical patient information and computer-executable instructions; and
processor hardware configured to execute the computer-executable instructions, wherein the computer-executable instructions include:
accessing historical patient information associated with a patient;
identifying a plurality of events in the historical patient information that satisfy one or more criteria;
processing, by a first large language model (LLM), the plurality of events that satisfy the one or more criteria, to generate a historical health output associated with the patient;
generating, for display in a graphical user interface, one or more interactive medical tiles associated with the plurality of events that satisfy the one or more criteria, according to the historical health output associated with the patient;
receiving a selection of one of the interactive medical tiles; and
presenting, in the graphical user interface, the plurality of events according to an organization criterion in response to receiving the selection of one of the interactive medical tiles.

2. The computer system of claim 1, wherein:
the one or more interactive medical tiles are displayed in a health summary format on the graphical user interface; and
the health summary format is specified according to the historical health output from the LLM.

3. The computer system of claim 2, wherein the health summary format includes at least one of:
a chatbot conversation textual output format;
a health journey timeline output format;
a periodic update textual output format; and
a social media update output format.

4. The computer system of claim 3, wherein the health summary format includes the chatbot conversation textual output format, and the chatbot conversation textual output format includes at least one health summary comment displayed in response to a user input prompt.

5. The computer system of claim 3, wherein the health summary format includes the health journey timeline output format, and the health journey timeline output format includes multiple health events displayed in consecutive timeline by event date, next to an electronic health record of the patient corresponding to the health event.

6. The computer system of claim 3, wherein the health summary format includes the periodic update textual output format, and the periodic update textual output format includes a summary one or more health events for the patient occurring within a last day, a last week and a last month.

7. The computer system of claim 1, accessing historical patient information associated with the patient includes accessing multiple electronic health records of the patient from an electronic health record database.

8. The computer system of claim 7, wherein accessing historical patient information associated with the patient includes accessing at least one of community and support data associated with the patient, data acquired from one or more wearable devices of the patient, environmental data corresponding to an environment of the patient, or demographic data of the patient.

9. The computer system of claim 8, wherein processing, by the first large language model (LLM), the plurality of events that satisfy the one or more criteria, includes assigning a higher weight to the multiple electronic health records of the patient compared to data obtained from other sources.

10. The computer system of claim 1, wherein processing, by the first large language model (LLM), the plurality of events that satisfy the one or more criteria, includes processing the plurality of events to determine at least one of a healthcare milestone achieved by the patient, a healthcare setback experienced by the patient, healthcare goal progress achieved by the patient, an ally support event associated with the patient, or a healthcare decision made by the patient.

11. The computer system of claim 1, wherein processing, by the first large language model (LLM), the plurality of events that satisfy the one or more criteria, includes processing the plurality of events to determine at least one of a healthcare need defined by the patient, a healthcare goal defined by the patient, a best practice related to healthcare for the patient, a provider guidance item for the patient, or a treatment plan for a health condition of the patient.

12. The computer system of claim 1, wherein the computer-executable instructions include:
receiving input from the patient that includes one or more keywords related to an intent associated with one or more of the plurality of events;
processing, by the first large language model (LLM), the input from the patient to generate a prompt for a second LLM; and
processing the prompt by the second LLM together with the historical patient information to generate a response to the input.

13. The computer system of claim 12, wherein the computer-executable instructions further include:
processing the historical patient information to predict a set of inquiries associated with the plurality of events; and
receiving, as part of the input, a selection of an individual inquiry of the set of inquiries.

14. The computer system of claim 12, wherein the first LLM comprises an artificial neural network, and wherein the second LLM comprises an artificial neural network.

15. The computer system of claim 12, wherein the input comprises a document inquiry, and the computer-executable instructions further include:
receiving a medical document as part of the input; and
processing the medical document by the second LLM to predict a set of intents associated with the medical document.

16. The computer system of claim 15, wherein the computer-executable instructions further include:
generating a query based on content of the medical document and an individual intent of the set of intents;
obtaining information corresponding to the query; and
presenting the information in the graphical user interface.

17. The computer system of claim 1, wherein the computer-executable instructions further include processing the historical patient information by an artificial neural network to select the plurality of events, the artificial neural network being trained using training data to identify events that satisfy the one or more criteria.

18. The computer system of claim 17, wherein the computer-executable instructions further include:
accessing training data comprising training patient information and corresponding ground truth collections of events in the training patient information that satisfy the one or more criteria;
processing, by the artificial neural network, the training patient information to estimate a plurality of events;
computing a deviation between the plurality of events and the corresponding ground truth collections of events; and
updating one or more parameters of the artificial neural network based on the computed deviation.

19. A method comprising:
accessing historical patient information associated with a patient;
identifying a plurality of events in the historical patient information that satisfy one or more criteria;
processing, by a first large language model (LLM), the plurality of events that satisfy the one or more criteria, to generate a historical health output associated with the patient;
generating, for display in a graphical user interface, one or more interactive medical tiles associated with the plurality of events that satisfy the one or more criteria, according to the historical health output associated with the patient;
receiving a selection of one of the interactive medical tiles; and
presenting, in the graphical user interface, the plurality of events according to an organization criterion in response to receiving the selection of one of the interactive medical tiles.

20. A non-transitory computer-readable medium comprising non-transitory computer-readable instructions for performing operations comprising:
accessing historical patient information associated with a patient;
identifying a plurality of events in the historical patient information that satisfy one or more criteria;
processing, by a first large language model (LLM), the plurality of events that satisfy the one or more criteria, to generate a historical health output associated with the patient;
generating, for display in a graphical user interface, one or more interactive medical tiles associated with the plurality of events that satisfy the one or more criteria, according to the historical health output associated with the patient;
receiving a selection of one of the interactive medical tiles; and
presenting, in the graphical user interface, the plurality of events according to an organization criterion in response to receiving the selection of one of the interactive medical tiles.
